(19) United States
(12) Patent Application Publication
(10) Pub. No.: US 2006/0004428 A1
(43) Pub. Date: Jan. 5, 2006

(54) Pyrrolo (3,4-c) carbazole and pyrido (2,3-b) pyrrolo (3,4-e) indole derivatives, preparation method and pharmaceutical compositions containing same

(76) Inventors: Michelle Prudhomme, Clermont-Ferrand (FR); Bernadette Hugon, Clermont-Ferrand (FR); Fabrice Anizon, Ennezat (FR); John Hickman, Paris (FR); Alain Pierre, Les Alluets Le Roi (FR); Roy Golsteyn, Maurecourt (FR); Pierre Renard, Le Chesnay (FR); Bruno Pfeiffer, Saint Leu La Foret (FR)

Correspondence Address: The Firm of Hueschen and Sage, 500 Columbia Plaza, 350 East Michigan Avenue, Kalamazoo, MI, 49007, US

(21) Appl. No.: 10/531,620

(22) PCT Filed: Oct. 14, 2003
(86) PCT No.: PCT/FR03/03021

§ 371 Date: Apr. 13, 2005

(30) Foreign Application Priority Data
Oct. 16, 2002 (FR) 02/12846

(51) Int. Cl. A61N 1/00

(52) U.S. Cl. 607/115

(57) ABSTRACT

A compound selected from those of formula (I): 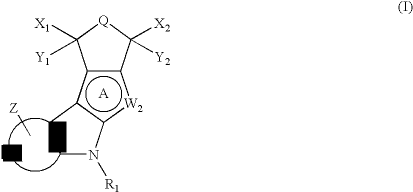 wherein: Z represents a group of the formula U-V as defined in the description,
W₁ represents together with carbon to which it is bonded, phenyl, pyridyl,
W₂ is as defined in the description,
X₁, X₂ each represents hydrogen, hydroxy, alkoxy, mercapto or alkylthio,
Y₁, Y₂ each represents hydrogen,
or X₁ and Y₁, X₂ and Y₂ with carbon carrying them, together form carbonyl or thiocarbonyl,
R₁ is as defined in the description,
Q represents oxygen, a group NR₂ as defined in the description, its isomers, and addition salts thereof with a pharmaceutically acceptable acid or base, and medicinal products containing the same which are useful in the treatment of cancer.

The present invention relates to new pyrrolo[3,4-c]carbazole and pyrido[2,3-b]pyrrolo-[3,4-e]indole compounds, to a process for their preparation and to pharmaceutical compositions containing them.

The needs of anti-cancer therapy call for the constant development of new anti-proliferative agents, with the aim of obtaining medicaments that are both more active and better tolerated. The compounds of the present invention have anti-tumour properties in particular, which accordingly render them useful in the treatment of cancers.

Among the types of cancers which can be treated with the compounds of the present invention there may be mentioned, without implying any limitation, adenocarcinomas and carcinomas, sarcomas, gliomas and leukaemias.

By virtue of their properties, the compounds of the invention can advantageously be associated with the totality of the cytotoxic treatments currently in use, as well as with radiotherapies, whose toxicity is not increased thereby, and with the various hormone therapies directed against cancers (breast and prostate).

Patent applications WO 95/07910 and WO 96/04906 describe indole compounds and claim them on the one hand for their anti-viral activity and on the other hand for the treatment and prevention of restenosis. Patent applications WO 00/47583, WO 97/21677 and WO 96/11933 disclose cyclopenta[g]pyrrolo[3,4-e]indole compounds which are fused on the indole moiety and the cyclopentene moiety of the compounds to an aromatic or non-aromatic ring system and which optionally contain hetero atoms. Those compounds have pharmacological activities which render them useful especially in the treatment of cancer. Patent application WO 01/85686 describes pyrrolo[3,4-c]carbazole compounds for use in the treatment of neurodegenerative diseases, inflammations, ischaemia and cancer.

The present invention relates more specifically to compounds of formula (I): 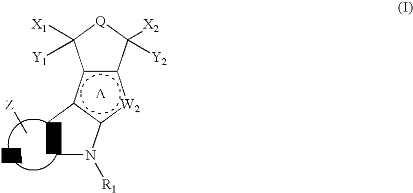
wherein:
A represents a ring having 6 ring members which is saturated or partially or wholly unsaturated, wherein the unsaturation optionally confers an aromatic nature on the ring,
Z represents one or more identical or different groups of the formula U-V wherein:
   U represents a single bond, or a linear or branched (C₁-C₆)alkylene chain which is optionally substituted by one or more identical or different groups selected from halogen and hydroxy and/or which optionally contains one or more unsaturated bonds,
   V represents a group selected from a hydrogen atom, a halogen atom and the groups cyano, nitro, azido, linear or branched (C₁-C₆)alkyl, aryl, aryl(C₁-C₆)alkyl in which the allyl moiety may be linear or branched, hydroxy, linear or branched (C₁-C₆)alkoxy, alkoxy, aryloxy, aryl(C₁-C₆)alkoxy in which the alkoxy moiety may be linear or branched, formyl, carboxy, aminocarbonyl, NR₃R₄, —C(O)-T₁, —C(O)—NR₃-T₁, —NR₃—C(O)-T₁, —O—C(O)-T₁, —C(O)—O-T₁, —O-T₂-NR₃R₄, —O-T₂-OR₃, —O-T₂-CO₂R₃, —NR₃-T₂-NR₃R₄, —NR₃-T₂-OR₃, —NR₃-T₂-CO₂R₃ and —S(O)ₜ—R₃, wherein:
      R₃ and R₄, which are identical or different, each represents a group selected from a hydrogen atom and the groups linear or branched (C₁-C₆)alkyl, aryl, and aryl(C₁-C₆)alkyl in which the alkyl moiety may be linear or branched, or
      R₃+R₄ form together, with the nitrogen atom carrying them, a saturated, monocyclic or bicyclic heterocycle which has from 5 to 10 ring atoms and optionally contains within the ring system a second hetero atom selected from oxygen and nitrogen and which is optionally substituted by a group selected from linear or branched (C₁-C₆)alkyl, aryl, aryl(C₁-C₆)alkyl in which the alkyl moiety may be linear or branched, hydroxy, linear or branched (C₁-C₆)alkoxy, amino, linear or branched mono(C₁-C₆)alkylamino, and di(C₁-C₆)alkylamino in which the alkyl moieties may be linear or branched,
      T₁ represents a group selected from linear or branched (C₁-C₆)alkyl optionally substituted by a group selected from —OR₃, —NR₃R₄, —CO₂R₃, —C(O)R₃ and —C(O)NR₃R wherein R₃ and R₄ are as defined hereinbefore, aryl, and aryl(C₁-C₆)alkyl in which the alkyl moiety may be linear or branched, or T, represents a linear or branched (C₂-C₆)alkenyl chain optionally substituted by a group selected from —OR₃, —NR₃R₄, —CO₂R₃, —C(O)R₃ and —C(O)NR₃R₄ wherein R₃ and R₄ are as defined hereinbefore,
   T₂ represents a linear or branched (C₁-C₆)alkylene chain,
   t represents an integer from 0 to 2 inclusive,
   or a methylenedioxy or ethylenedioxy group
W₁, with the carbon atoms to which it is bonded, represents a phenyl group or a pyridyl group,
W₂ represents a group selected from: 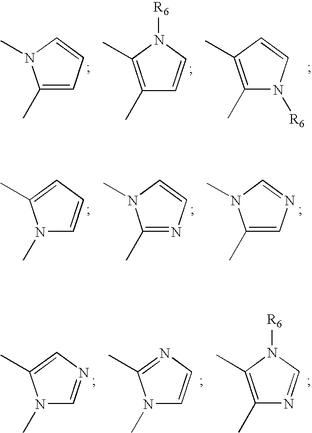
   wherein R₆ represents a group selected from a hydrogen atom and the groups linear or branched (C₁-C₆)alkyl, aryl, aryl(C₁-C₆)alkyl in which the alkyl moiety may be linear or branched, cycloalkyl, cycloalkyl(C₁-C₆)alkyl in which the alkyl moiety may be linear or branched, —OR₃, —NR₃R₄, —O-T₂-NR₃R₄, NR₃-T₂-NR₃R₄, linear or branched (C₁-C₆)hydroxyalkylamino, di((C₁-C₆)hydroxyalkyl)amino in which the alkyl moieties may be linear or branched, —C(O)R₃ and —NH—C(O)—R₃, or R₆ represents a linear or branched (C₁-C₆)alkylene chain substituted by one or more identical or different groups selected from halogen atoms and the groups cyano, nitro, —OR₃, —NR₃R₄, —CO₂R₃, —C(O)R₃, linear or branched (C₁-C₆)hydroxyalkylamino, di((C₁-C₆)hydroxyalkyl)amino in which the alkyl moieties may be linear or branched, and —C(O)—NHR₃, the groups R₃, R₄ and T₂ being as defined hereinbefore,
X₁ represents a group selected from a hydrogen atom and the groups hydroxy, linear or branched (C₁-C₆)alkoxy, mercapto and linear or branched (C₁-C₆)alkylthio,
Y₁ represents a hydrogen atom, or
X₁ and Y₁ form together, with the carbon atom carrying them, a carbonyl or thiocarbonyl group,
X₂ represents a group selected from a hydrogen atom and the groups hydroxy, linear or branched (C₁-C₆)alkoxy, mercapto and linear or branched (C₁-C₆)alkylthio,
Y₂ represents a hydrogen atom, or
X₂ and Y₂ form together, with the carbon atom carrying them, a carbonyl or thiocarbonyl group,
R₁ represents a group selected from a hydrogen atom, a linear or branched (C₁-C₆)alkyl group optionally substituted by one or more groups hydroxy, linear or branched (C₁-C₆)alkoxy, linear or branched (C₁-C₆)hydroxyalkoxy or NR₃R₄, the groups R₃ and R₄ being as defined hereinbefore, or R₁ represents a group of the formula C(O)—O-T₃ wherein: T₃ represents a linear or branched (C₁-C₆)alkyl group, an aryl group or an aryl(C₁-C₆)alkyl group in which the alkyl moiety may be linear or branched, or R₁ represents a group of formula (a): 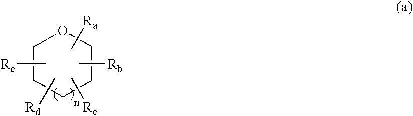
   wherein:
Rₐ, R_{b}, R_{c} and R_{d}, which are identical or different, each independently of the others represents a bond or a group selected from a hydrogen atom, a halogen atom and the groups hydroxy, linear or branched (C₁-C₆)alkoxy, aryloxy, aryl(C₁-C₆)alkoxy in which the alkoxy moiety may be linear or branched, linear or branched (C₁-C₆)alkyl, aryl(C₁-C₆)alkyl in which the alkyl moiety may be linear or branched, aryl, —NR₃R₄ wherein R₃ and R₄ are as defined hereinbefore, azido, —N═NR₃ (wherein R₃ is as defined hereinbefore), and —O—C(O)—R₅ wherein R₅ represents a linear or branched (C₁-C₆)alkyl group (optionally substituted by one or more groups selected from halogen, hydroxy, amino, linear or branched (C₁-C₆)alkylamino and di(C₁-C₆)alkylamino in which the alkyl moieties may be linear or branched), or R₅ represents aryl, aryl(C₁-C₆)alkyl in which the alkyl moiety may be linear or branched, cycloalkyl or heterocycloalkyl,
Rₑ represents a methylene group (H₂C═) or a group of the formula —U₁—Rₐ wherein U₁ represents a single bond or a methylene group and Rₐ is as defined hereinbefore,
n has the value 0 or 1,
   it being understood that the group of formula (a) is bonded to the nitrogen atom by Rₐ, R_{b}, R_{c}, R_{d} or Rₑ,
Q represents a group selected from an oxygen atom and a group NR₂ wherein R₂ represents a group selected from a hydrogen atom and the groups linear or branched (C₁-C₆)alkyl, aryl, aryl(C₁-C₆)alkyl in which the alkyl moiety may be linear or branched, cycloalkyl, cycloalkyl(C₁-C₆)alkyl in which the alkyl moiety may be linear or branched, —OR₃, —NR₃R₄, —O-T₂-NR₃R₄, —NR₃-T₂NR₃R₄, linear or branched (C₁-C₆)hydroxyalkylamino, di((C₁-C₆)hydroxyalkyl)amino in which the alkyl moieties may be linear or branched, —C(O)—R₃ and —NH—C(O)—R₃, or R₂ represents a linear or branched (C₁-C₆)alkylene chain substituted by one or more identical or different groups selected from halogen atoms and the groups cyano, nitro, —OR₃, —NR₃R₄, —CO₂R₃, —C(O)R₃, linear or branched (C₁-C₆)hydroxyalkylamino, di((C₁-C₆)hydroxyalkyl)amino in which the alkyl moieties may be linear or branched, and —C(O)—NHR₃, the groups R₃, R₄ and T₂ being as defined hereinbefore,
   provided that when W₁, with the carbon atoms to which it is bonded, represents an unsubstituted phenyl group or a phenyl group substituted by a bromine atom, R₁ represents a group selected from a hydrogen atom and a glucopyranosyl or (2,3,4,6-tetra-O-benzyl-glucopyranosyl) group and R₂ represents a hydrogen atom, then W₂ represents a group selected from: 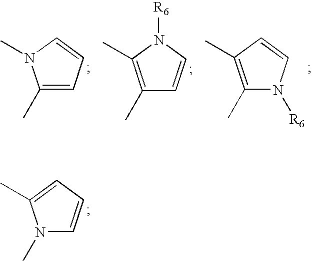
   wherein R₆ is as defined hereinbefore,
   and provided also that when W₁, with the carbon atoms to which it is bonded, represents an unsubstituted phenyl group, R₁ represents a hydrogen atom and R₂ represents a methyl group, then W₂ represents a group selected from: 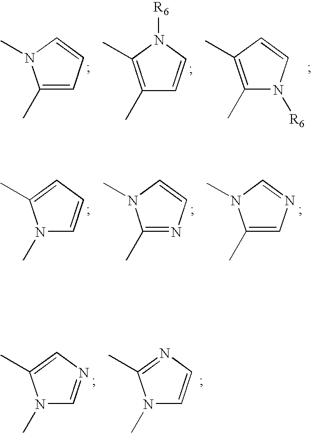
   wherein R₆ is as defined hereinbefore,
   to their enantiomers, diastereoisomers and also to addition salts thereof with a pharmaceutically acceptable acid or base,
   aryl being understood to be a phenyl, naphthyl, dihydronaphthyl, tetrahydronaphthyl, indenyl or indanyl group, each of those groups being optionally substituted by one or more identical or different groups selected from halogen, linear or branched (C₁-C₆)alkyl, linear or branched (C₁-C₆)trihaloalkyl, hydroxy, linear or branched (C₁-C₆)alkoxy, and NR₃R₄, R₃ and R₄ being as defined hereinbefore.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine etc.

Preferred compounds of the invention are those wherein X₁ and Y₁, with the carbon atom carrying them, together form a carbonyl group, and X₂ and Y₂, with the carbon atom carrying them, together form a carbonyl group.

In an embodiment of interest, the group Q to which preference is given in accordance with the invention is a group NR₂ wherein R₂ is as defined for formula (I).

According to an advantageous embodiment, preferred compounds of the invention are compounds of formula (I) that correspond more especially to formula (IA): 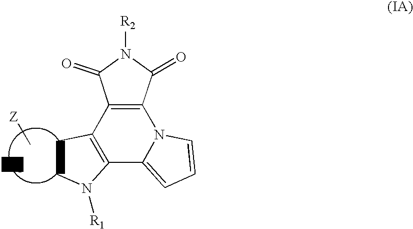
wherein R₁, R₂, W₁ and Z are as defined for formula (I).

According to a second advantageous embodiment, preferred compounds of the invention are compounds of formula (I) that correspond more especially to formula (IB): 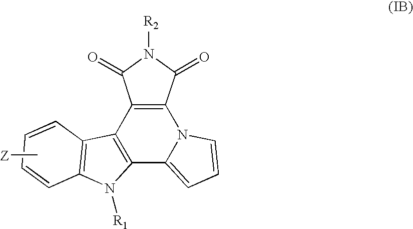
wherein R₁, R₂ and Z are as defined for formula (I).

According to a third advantageous embodiment, preferred compounds of the invention are compounds of formula (O) that correspond more especially to formula (IC): 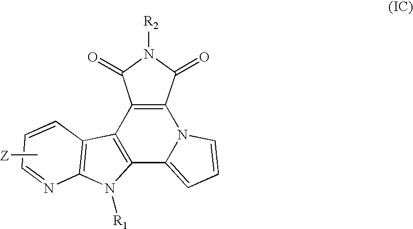
wherein R₁, R₂ and Z are as defined for formula (I).

According to a fourth advantageous embodiment, preferred compounds of the invention are compounds of formula (I) that correspond more especially to formula (ID): 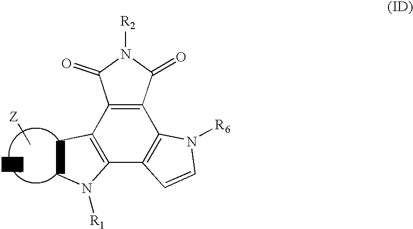
wherein R₁, R₂, R₆, W₁ and Z are as defined for formula (I).

According to a fifth advantageous embodiment, preferred compounds of the invention are compounds of formula (I) that correspond more especially to formula (IE): 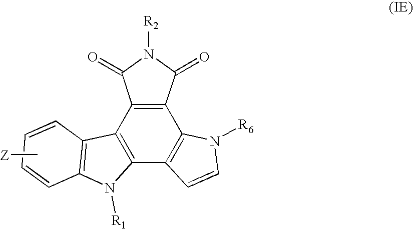
wherein R₁, R₂, R₆ and Z are as defined for formula (I).

According to a sixth advantageous embodiment, preferred compounds of the invention are compounds of formula (I) that correspond more especially to formula (IF): 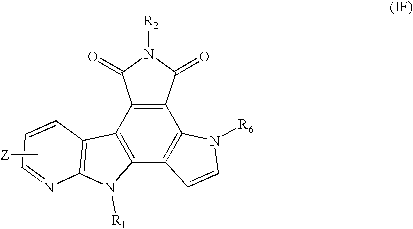
wherein R₁, R₂, R₆ and Z are as defined for formula (I).

According to a seventh advantageous embodiment, preferred compounds of the invention are compounds of formula (I) that correspond more especially to formula (IG): 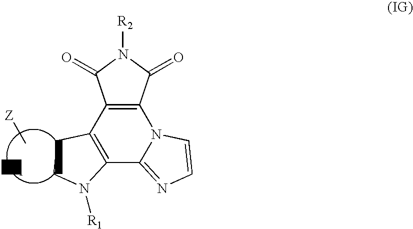
wherein R₁, R₂, W₁ and Z are as defined for formula (I).

According to an eighth advantageous embodiment, preferred compounds of the invention are compounds of formula (I) that correspond more especially to formula (IH): 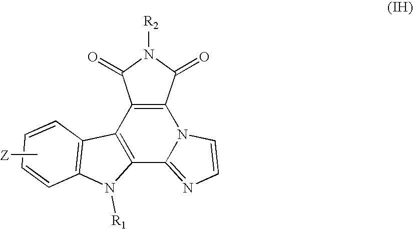
wherein R₁, R₂ and Z are as defined for formula (I).

According to a ninth advantageous embodiment, preferred compounds of the invention are compounds of formula (I) that correspond more especially to formula (II): 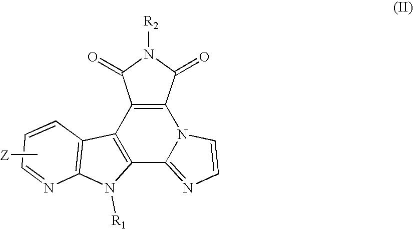
wherein R₁, R₂ and Z are as defined for formula (I).

According to a tenth advantageous embodiment, preferred compounds of the invention are compounds of formula (I) that correspond more especially to formula (IJ): 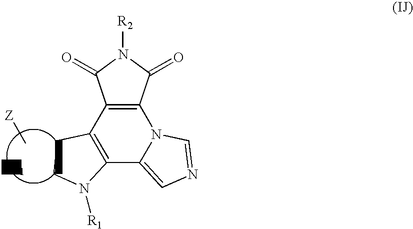
wherein R₁, R₂, W₁ and Z are as defined for formula (I).

According to an eleventh advantageous embodiment, preferred compounds of the invention are compounds of formula (I) that correspond more especially to formula (IK): 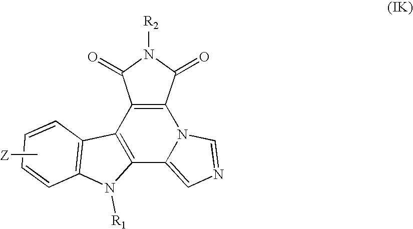
wherein R₁, R₂ and Z are as defined for formula (I).

According to a twelfth advantageous embodiment, preferred compounds of the invention are compounds of formula (I) that correspond more especially to formula (IL): 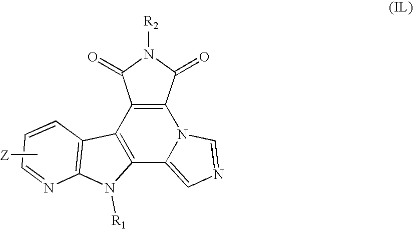
wherein R₁, R₂ and Z are as defined for formula (I).

Advantageously, the group R₁ to which preference is given in accordance with the invention is a hydrogen atom, a group of the formula C(O)—O-T₃ wherein T₃ represents a linaer or branched (C₁-C₆)alkyl group, or a glucopyranosyl group of the formula:

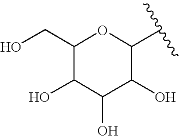

In an embodiment of interest, the group R₂ to which preference is given in accordance with the invention is a hydrogen atom or a linear or branched (C₁-C₆)alkyl group.

Advantageously, the group R₆ to which preference is given in accordance with the invention is a hydrogen atom.

Compounds of the invention to which preference is given are:
pyrrolo[3′,4′:5,6]indolizino[8,7-b]indole-1,3 [2H,8H]-dione,
11-bromopyrrolo[3′,4′:5,6]indolizino[8,7-b]indole-1,3 [2H,8H]-dione,
11-chloropyrrolo[3′,4′:5,6]indolizino[8,7-b]indole-1,3[2H,8H]-dione,
imidazo[2′, 1′:6,1]pyrrolo[3′,4′:4,5]pyrido[2,3-b]indole-1,3 (2H,8H)-dione.

The enantiomers, diastereoisomers and addition salts with a pharmaceutically acceptable acid or base of the preferred compounds form an integral part of the invention.

The present invention relates also to a process for the preparation of compounds of formula (I), characterised in that there is used as starting material a compound of formula (II): 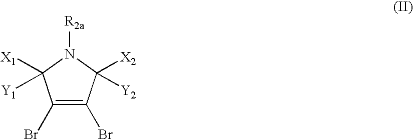
wherein R₂ₐ represents a hydrogen atom or a methyl group and X₁, Y₁, X₂ and Y₂ are as defined for formula (I),
which compound of formula (II) is treated with an alkylmagnesium halide in the presence of a compound of formula (III): 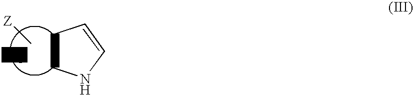
wherein W₁ and Z are as defined for formula (I), to yield a compound of formula (IV): 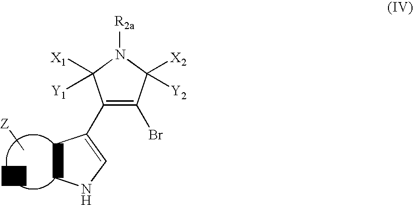
wherein R₂ₐ, X₁, Y₁, X₂, Y₂, W₁ and Z are as defined hereinbefore,
which compound of formula (IV) is reacted with di-tert-butyl dicarbonate in the presence of 4-dimethylaminopyridine to yield a compound of formula (V): 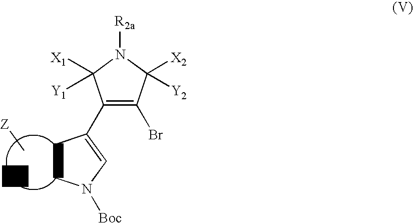
wherein Boc represents a tert-butylearbonyloxy group and R₂ₐ, X₁, Y₁, X₂, Y₂, W₁ and Z are as defined hereinbefore,
which compound of formula (V) is:
either treated with an alkylmagnesium halide in the presence of a pyrrolyl compound to yield a compound of formula (VI): 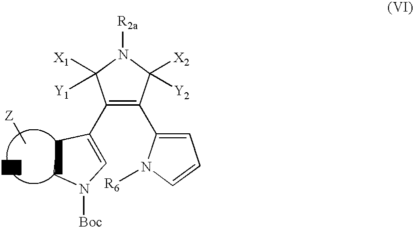
   wherein R₆ is as defined for formula (I) and Boc, R₂ₐ, X₁, Y₁, X₂, Y₂, W₁ and Z are as defined hereinbefore,
   which compound of formula (VI) is:
either irradiated with a halogen lamp to yield a compound of formula (I/a), which is a particular case of the compounds of formula (I): 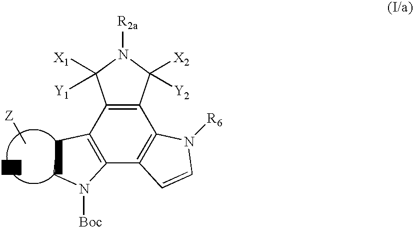
   wherein R₆, R₂ₐ, X₁, Y₁, X₂, Y₂, W₁ and Z are as defined hereinbefore,
   which compound of formula (I/a) is optionally treated with formic acid to yield a compound of formula (I/b), which is a particular case of the compounds of formula (I): 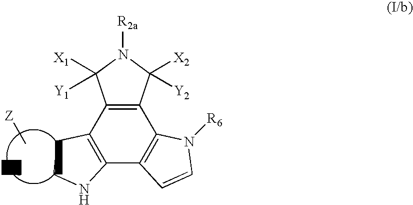
   wherein R₆, R₂ₐ, X₁, Y₁, X₂, Y₂, W₁ and Z are as defined hereinbefore,
or treated with palladium black in the particular case where R₆ represents a hydrogen atom, to yield a compound of formula (I/c), which is a particular case of the compounds of formula (I): 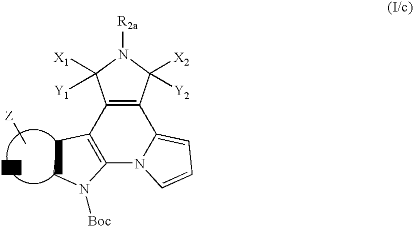
   wherein Boc, R₂ₐ, X₁, Y₁, X₂, Y₂, W₁ and Z are as defined hereinbefore, which compound of formula (I/c) is optionally subjected to the same reaction conditions as the compound of formula (I/a) to yield a compound of formula (I/d), which is a particular case of the compounds of formula (I): 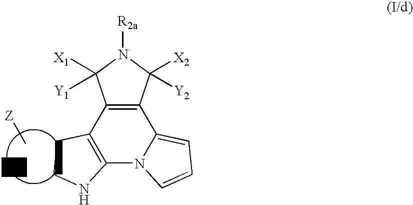
   wherein R₂ₐ, X₁, Y₁, X₂, Y₂, W₁ and Z are as defined hereinbefore,
or treated with lithium hexamethyldisilazane in the presence of a pyrrolyl compound to yield a compound of formula (VII): 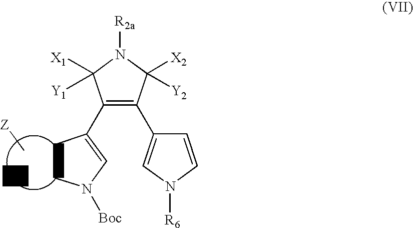
   wherein Boc, R₆, R₂ₐ, X₁, Y₁, X₂, Y₂, W₁ and Z are as defined hereinbefore,
   which compound of formula (VII) is irradiated with a halogen lamp, in an apolar and aprotic solvent, to yield a compound of formula (I/e), which is a particular case of the compounds of formula (I): 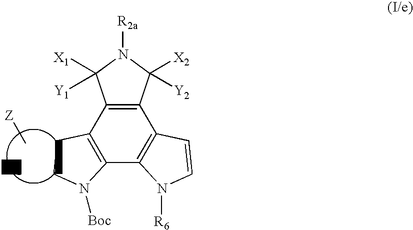
   wherein Boc, R₆, R₂ₐ, X₁, Y₁, X₂, Y₂, W₁ and Z are as defined hereinbefore, which compound of formula (I/e) is optionally subjected to the same reaction conditions as the compound of formula (I/a) to yield a compound of formula (I/f), which is a particular case of the compounds of formula (I): 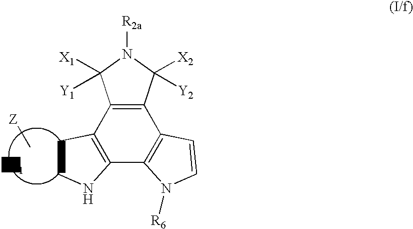
   wherein R₆, R₂ₐ, X₁, Y₁, X₂, Y₂, W₁ and Z a as defined hereinbefore,
or treated with an alkylmagnesium halide in the presence of imidazole to yield a compound of formula (VIII): 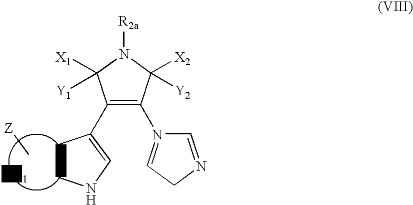
   wherein R₂ₐ, X₁, Y₁, X₂, Y₂, W₁ and Z are as defined hereinbefore,
   which compound of formula (VIII) is treated with a compound of formula (IX):
   R₁ₐ-G (IX)
   wherein R₁ₐ, which is other than a hydrogen atom, has the same definition as R₁ in formula (I) and G represents a hydroxy group or a leaving group,
   to yield a compound of formula (X): 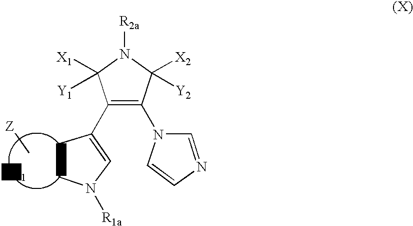
   wherein R₁ₐ, R₂ₐ, X₁, Y₁, X₂, Y₂, W₁ and Z are as defined hereinbefore,
   which compounds of formula (X) are irradiated with a halogen lamp to yield compounds of formulae (I/g₁) and (I/g₂), which are particular cases of the compounds of formula (I): 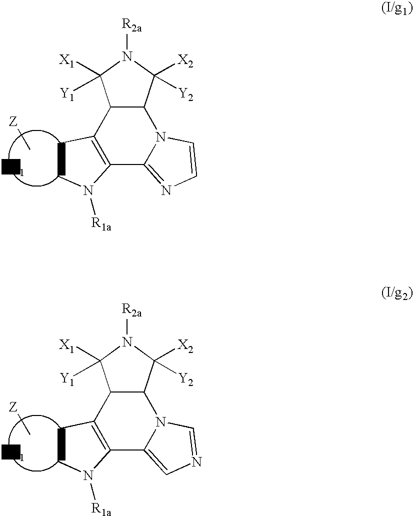
   wherein R₁ₐ, R₂ₐ, X₁, Y₁, X₂, Y₂, W₁ and Z are as defined hereinbefore,
   which compounds of formulae (I/g₁) and (I/g₂) are optionally treated with manganese dioxide to yield compounds of formulae (I/h₁) and (I/h₂), which are particular cases of the compounds of formula (I): 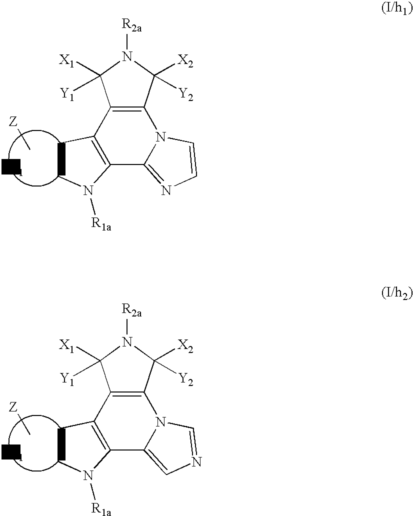
   wherein R₁ₐ, R₂ₐ, X₁, Y₁, X₂, Y₂, W₁ and Z are as defined hereinbefore,
   which compounds of formulae (I/h₁) and (I/h₂) are optionally subjected to the same reaction conditions as the compound of formula (I/a), in the particular case where R₁ₐ represents a tert-butylcarbonyloxy group, to yield compounds of formulae (I/i₁) and (I/i₂), which are particular cases of the compounds of formula (I): 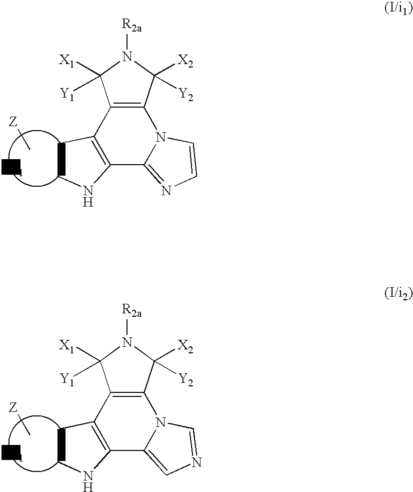
   wherein R₂ₐ, X₁, Y₁, X₂, Y₂, W₁ and Z are as defined hereinbefore,
or treated with an alkylmagnesium halide in the presence of an imidazolyl compound (XI): 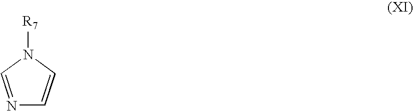
   wherein R₇ represents a secondary-amine-protecting group known to the person skilled in the art, to yield a compound of formula (XII): 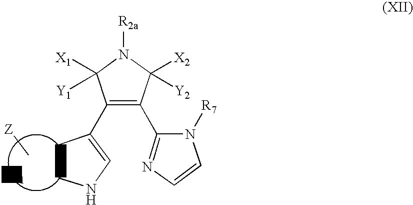
   wherein R₂ₐ, R₇, X₁, Y₁, X₂, Y₂, W₁ and Z are as defined hereinbefore,
   which compound of formula (XII) is subjected to the same reaction conditions as the compound of formula (VIII) to yield a compound of formula (XIII): 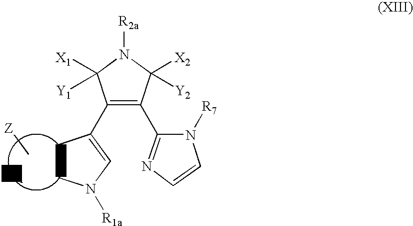
   wherein R₁ₐ, R₂ₐ, R₇, X₁, Y₁, X₂, Y₂, W₁ and Z are as defined hereinbefore,
   in which compound of formula (XIII) the imidazolyl ring is deprotected by conventional methods of organic synthesis known to the person skilled in the art to yield a compound of formula (XIV): 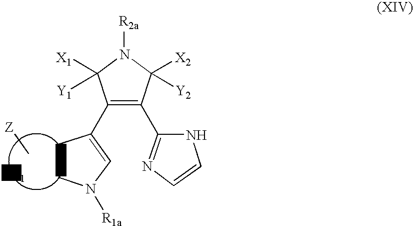
   wherein R₁ₐ, R₂ₐ, X₁, Y₁, X₂, Y₂, W₁ and Z are as defined hereinbefore,
   which compound of formula (XIV) is treated with palladium black to yield a compound of formula (I/j), which is a particular case of the compounds of formula (I): 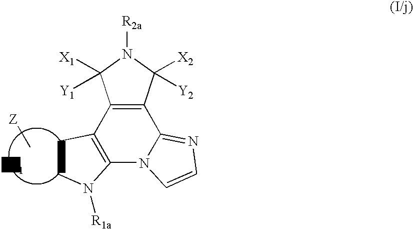
   wherein R₁ₐ, R₂ₐ, X₁, Y₁, X₂, Y₂, W₁ and Z are as defined hereinbefore,
   which compound of formula (I/j) is optionally subjected to the same reaction conditions as the compounds of formula (I/h) to yield a compound of formula (I/k), which is a particular case of the compounds of formula (I): 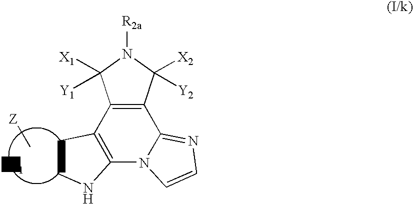
   wherein R₂ₐ, X₁, Y₁, X₂, Y₂, W₁ and Z are as defined hereinbefore,
or treated with an alkylmagnesium halide in the presence of an imidazolyl compound (XV): 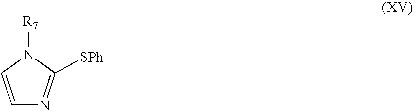
   wherein R₇ is as defined hereinbefore, to yield a compound of formula (XVI): 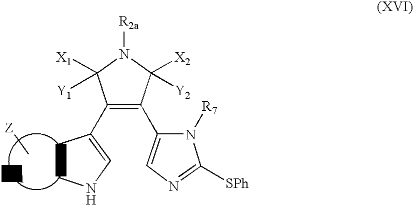
   wherein R₂ₐ, R₇, X₁, Y₁. X₂, Y₂, W₁ and Z are as defined hereinbefore,
   which compound of formula (XVI) is treated with Raney nickel to yield a compound of formula (XVII): 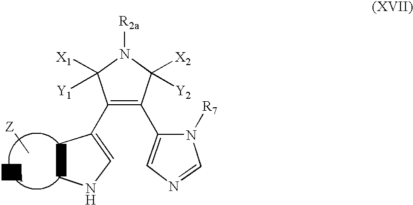
   wherein R₂ₐ, R₇, X₁, Y₁, X₂, Y₂, W₁ and Z are as defined hereinbefore,
   which compound of formula (XVII) is subjected in succession to the same reaction conditions as the compounds of formulae (XII) and (XIII) to yield a compound of formula (XVIII): 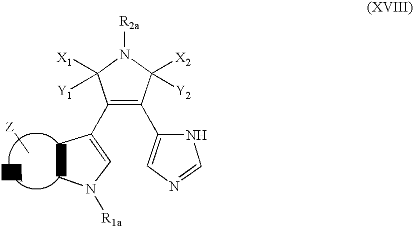
   wherein R₁ₐ, R₂ₐ, X₁, Y₁, X₂, Y₂, W₁ and Z are as defined hereinbefore,
   which compound of formula (XVIII) is:
either irradiated with a halogen lamp in the presence of palladium-on-carbon to yield a compound of formula (I/l), which is a particular case of the compounds of formula (I): 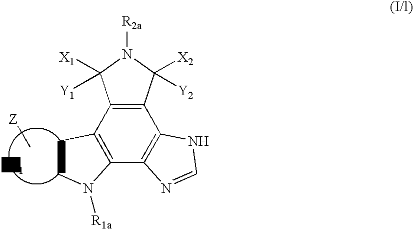
   wherein R₁ₐ, R₂ₐ, X₁, Y₁, X₂, Y₂, W₁ and Z are as defined hereinbefore,
   which compound of formula (I/l) is optionally subjected to the same reaction conditions as the compounds of formula (I/h) to yield the compounds of formula (I/m), which are a particular case of the compounds of formula (I): 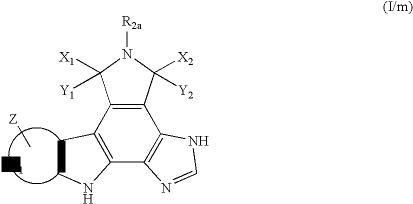
   wherein R₂ₐ, X₁, Y₁, X₂, Y₂, W₁ and Z are as defined hereinbefore,
or subjected to the same reaction conditions as the compound of formula (XIV) to yield the compounds of formula (I/n), which are a particular case of the compounds of formula (I): 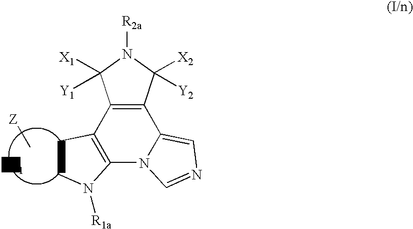
   wherein R₁ₐ, R₂ₐ, X₁, Y₁, X₂, Y₂, W₁ and Z are as defined hereinbefore,
   which compounds of formula (I/n) are optionally subjected to the same reaction conditions as the compounds of formula (I/l) to yield the compounds of formula (I/o), which are a particular case of the compounds of formula (I): 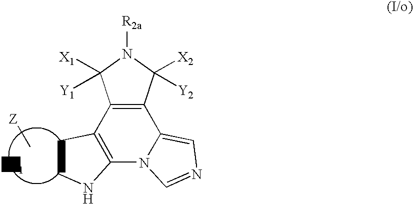
   wherein R₂ₐ, X₁, Y₁, X₂, Y₂, W₁ and Z are as defined hereinbefore,
   the compounds of formulae (I/a) to (I/o) constituting the compounds of formula (I/p): 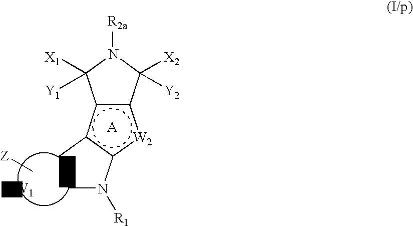
   wherein A, R₁, R₂ₐ, X₁, Y₁, X₂, Y₂, W₁ and Z are as defined hereinbefore,
   which compound of formula (I/p) is optionally treated with aqueous sodium hydroxide and then placed in the presence of hydrochloric acid to yield a compound of formula (I/q), which is a particular case of the compounds of formula (I): 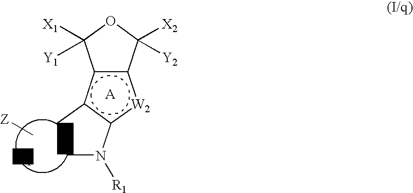
   wherein A, R₁, X₁, Y₁, X₂, Y₂, W₁, W₂ and Z are as defined hereinbefore,
   which compound of formula (I/q) is optionally treated with a compound of formula (XIX):
   R_{2b}—NH₂ (XIX)
   wherein R_{2b}, which is other than a hydrogen atom and a methyl group, is as defined for R₂ in formula (I), to yield a compound of formula (I/r), which is a particular case of the compounds of formula (I): 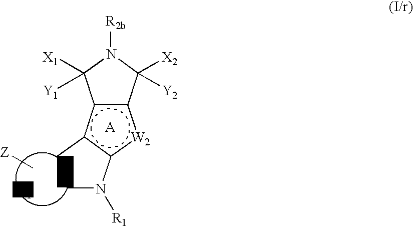
   wherein A, R₁, R_{2b}, X₁, Y₁, X₂, Y₂, W₁, W₂ and Z are as defined hereinbefore,
   which compounds of formulae (I/a) to (1/r) constitute the totality of the compounds of formula (I), which are purified, where necessary, according to conventional purification techniques, which may be separated, it desired, into their different isomers according to a conventional separation technique, and which are converted, if desired, into their addition-salts with a pharmaceutically acceptable acid or base.

According to an embodiment of the invention, the compounds of formula (I) wherein W₂ has the particular definition: 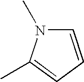
can be prepared starting from a compound of formula (XX): 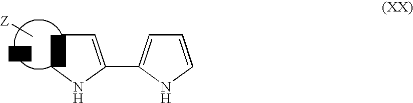
wherein W₁ and Z are as defined for formula (I),
which compounds of formula (XX) are reacted with a compound of formula (XXI): 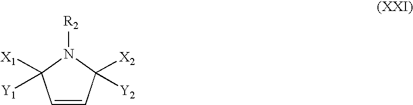
wherein R₂, X₁, Y₁, X₂ and Y₂ are as defined for formula (I),
to yield a compound of formula (XXII): 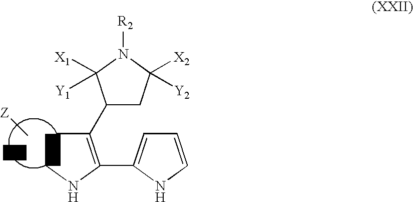
wherein R₂, X₁, Y₁, X₂, Y₂, W₁ and Z are as defined hereinbefore,
which compound of formula (XXII) is treated with palladium-on-carbon to yield a compound of formula (I/s), which is a particular case of the compounds of formula (I): 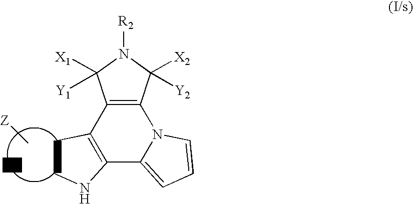
wherein R₂, X₁, Y₁, X₂, Y₂, W₁ and Z are as defined hereinbefore,
which compound of formula (I/s) is purified, where necessary, according to conventional purification techniques, may be separated, if desired, into its different isomers according to a conventional separation technique and is converted, if desired, into its addition salts with a pharmaceutically acceptable acid or base.

The compounds of formulae (II), (III), (IX), (XI), (XV), (XIX), (XX) and (XXI) are either commercially available compounds or are obtained according to conventional methods of organic synthesis which are readily accessible to the person skilled in the art.

The compounds of formula (I) have especially valuable anti-tumour properties. The properties that are characteristic of the compounds allow them to be used in therapeutics as anti-tumour agents.

The compounds of the invention can also be used in therapeutic association with another anti-cancer agent such as, for example, paclitaxel, tamoxifen and its derivatives, cisplatin and its analogues, irinotecan and its metabolites, the various alkylating agents of which the leader is cyclophosphamide, etoposide, the vinca alkaloids, doxorubicin and other anthracyclins and nitrosoureas.

The present invention relates also to pharmaceutical compositions comprising as active ingredient at least one compound of formula (I), its optical isomers or an addition salt thereof with a pharmaceutically acceptable base or acid, alone or in combination with one or more inert, non-toxic, pharmaceutically acceptable excipients or carriers.

Among the pharmaceutical compositions according to the invention there may be mentioned more especially those that are suitable for oral, parenteral (intravenous, intramuscular or subcutaneous), per- or trans-cutaneous, nasal, rectal, perlingual, ocular or respiratory administration, and especially tablets or dragées, sublingual tablets, gelatin capsules, capsules, suppositories, creams, ointments, dermal gels, injectable or drinkable preparations, aerosols, eye drops and nose drops, etc.

By virtue of the pharmacological properties that are characteristic of the compounds of formula (I), the pharmaceutical compositions comprising the said compounds of formula (I) as active ingredient are, accordingly, especially useful in the treatment of cancers.

The useful dose varies according to the age and weight of the patient, the administration route, the nature and severity of the disorder and any associated treatments and ranges from 1 mg to 500 mg per day, in one or more administrations.

The Examples that follow illustrate the invention, without limiting it in any way. The starting materials used are products which are known or are prepared according to known procedures.

The structures of the compounds described in the Examples were determined according to customary spectrophotometric techniques (infra-red, nuclear magnetic resonance, mass spectrometry, . . . )

### PREPARATION A: 2-(1H-Pyrrol-2-yl)-1H-indole

The expected product is obtained according to the process described by V. Bocchi et al. (Tetrahedron, 1984, 40, pp. 3251-3256).

### PREPARATION B: 5-(Benzyloxy)-2-(1H-pyrrol-2-yl)-1H-indole

### Step A: 5-(Benzyloxy)-3-bromo-1H-indole

A solution of bromine (4 mmol) in 20 ml of dimethylformamide is added dropwise to a solution of 5-benzyloxyindole (4 mmol) in 20 ml of dimethylformamide. The mixture is stirred at ambient temperature for 24 hours with the exclusion of light. The crude reaction mixture is poured into 200 ml of ice-water containing 1 ml of ammonium hydroxide and 0.2 ml of sodium thiosulphate. The expected product is obtained by crystallisation, filtration over a frit and then washing with distilled water.

Melting point: 89-92° C.

IR (KBr): ν_{NH}=3420 cm⁻¹

Mass spectrum (FAB): 301.01 [M⁺π

### Step B: 5-(Benzyloxy)-2-(1H-pyrrol-2-yl)-1H-indole

To a solution of the compound obtained in the preceding Step (1.5 mmol) dissolved in 8 ml of anhydrous dichloromethane there is added a solution of pyrrole (1.5 mmol) dissolved in 7 ml of anhydrous dichloromethane, followed by trifluoroacetic acid (45 μl). The mixture is stirred at ambient temperature for 4 hours. The solution is rendered basic with several drops of ammonium hydroxide and then evaporated to dryness. Purification by chromatography on silica gel (ethyl acetate/cyclohexane:2/8) yields the expected product.

Melting point: 178-182° C.

IR (KBr): ν_{NH}=3380-3420 cm⁻¹

Mass spectrum (FAB): 289.13 [M+H⁺]

### PREPARATION C: 5-Bromo-2-(1H-pyrrol-2-yl)-1H-indole

The expected product is obtained according to the process described in Preparation B, starting from 5-bromo-indole.

Melting point: 245° C.

IR (KBr): ν_{NH}=3400, 3410 cm⁻¹

Mass spectrum (FAB): 259.99 [M⁺]

### PREPARATION D 5-Chloro-2-(1H-pyrrol-2-yl)-1H-indole

The expected product is obtained according to the process described in Preparation B, starting from 5-chloro-indole.

Melting point: 223-227° C.

IR (KBr): ν_{NH}=3400, 3420 cm¹

Mass spectrum (FAB): 217.05 [M+H⁺]

### PREPARATION E: Tert-butyl 3-(4-bromo-1-methyl-2,5-dioxo-2,5-dihydro-1H-pyrrol-3-yl)-1H-indole-1-carboxylate

### Step A: 3-Bromo-4-(1H-indol-3-yl)-1-methyl-1H-pyrrole-2,5-dione

A solution containing 1.445 g of indole dissolved in 29 ml of dry tetrahydrofuran is brought to from −20 to −10° C., under argon, and then 26 ml of LiHMDS (1 M in hexane) are added dropwise in the course of 15 minutes. After 45 minutes at −10° C., the solution is diluted with a further 15 ml of tetrahydrofuran, and a solution containing 2 g of N-methyl-2,3-dibromomaleimide dissolved in 17 ml of tetrahydrofuran is added dropwise in the course of 30 minutes. After 15 minutes at −10° C. and 15 minutes at 0° C., the reaction is stopped by the addition at 0° C. of 50 ml of a 0.3 N hydrochloric acid solution. The reaction mixture is extracted with ethyl acetate and the organic phases are washed with a saturated NaCl solution, dried over MgSO₄ and then evaporated under reduced pressure. The desired product is precipitated with methanol.

Melting point: 167-168° C.

### Step B: Tert-butyl 3-(4-bromo-1-methyl-2,5-dioxo-2,5-dihydro-1H-pyrrol-3-yl)-1H-indole-1-carboxylate

A solution, under an inert atmosphere, containing 1 g of the product obtained in Step A, 30 mg of 4-dimethylaminopyridine, 1.58 g of di-tert-butyl dicarbonate and 15 ml of dry tetrahydrofuran is stirrred at ambient temperature for 24 hours. After removal of the solvents under reduced pressure, the crude reaction mixture is purified by chromatography on silica gel (petroleum ether/ethyl acetate/triethylamine:8/2/1%), allowing the expected product to be isolated.

Melting point: 137-138° C.

### PREPARATION F: Tert-butyl 3-(4-bromo-1-methyl-2,5-dioxo-2,5-dihydro-1H-pyrrol-3-yl)-1H-pyrrolo[2,3-b]pyridine-1-carboxylate

### Step A: 3-Bromo-1-methyl-4-(1H-pyrrolo[2,3-b]pyridin-3-yl)-1H-pyrrole-2,5-dione

A solution of ethylmagnesium bromide is prepared from magnesium (12.7 mmol) in suspension in bromoethane (12.7 mmol) and dry tetrahydrofuran (5 ml). The solution is stirred for one hour at ambient temperature, and then 7-azaindole (12.7 mmol) dissolved in 40 ml of anhydrous toluene is added dropwise. After 1.5 hours' stirring at ambient temperature, a solution of N-methyl-2,3-dibromomaleimide (3.53 mmol) in 40 ml of anhydrous toluene is added dropwise. After 20 minutes, 60 ml of dry dichloromethane are added and then the reaction mixture is stirred for 75 hours at 40° C. and then hydrolysed with a saturated aqueous ammonium chloride solution. The organic product is extracted with ethyl acetate, and then the organic phases are combined, dried over magnesium sulphate and filtered. After evaporation of the solvent and purification of the residue by chromatography on silica gel (cyclohexane/ethyl acetate:3/2), the expected product is isolated.

Melting point: 158° C.

### Step B: Tert-butyl 3-(4-bromo-1-methyl-2,5-dioxo-2,5-dihydro-1H-pyrrol-3-yl)-1H-pyrrolo[2,3-b]pyridine-1-carboxylate

The expected product is obtained according to the process described in Step B of Preparation E, starting from the compound described in the preceding Step.

Melting point: 102-103° C.

IR (KBr): ν_{C═O}=1710, 1740, 1770 cm⁻¹

### PREPARATION G: 2-(1H-Pyrrol-2-yl)-1H-pyrrolo[2,3-b]pyridine

A 2M solution of butyllithium in cyclohexane (25 mmol) is added to a solution of N,N-diisopropylamine (25 mmol) in 30 ml of tetrahydrofuran at 0° C. 3-Methylpyridine (5.35 mmol) is added to 16 mmol of that lithium N,N-diisopropylamine solution. The reaction mixture is stirred for 10 minutes at 0° C. and then brought to −78° C., prior to the addition of 2-cyanopyrrole (5.35 mmol). The temperature is raised to 0° C. over a period of 1.5 hours, prior to the addition of the remainder of the lithium N,N-diisopropylamine solution (9 mmol). The reaction mixture is then heated at 45° C. for 5 hours. After the mixture has returned to ambient temperature, water and then a saturated aqueous sodium chloride solution are added. The mixture is extracted with ethyl acetate, and the organic phase is dried over magnesium sulphate, filtered and then concentrated. Purification by column chromatography on silica gel (ethyl acetate/cyclohexane:6/4) yields the expected product.

Melting point: >150° C. (decomposition)

IR (KBr): ν_{NH}=3420 cm⁻¹

### EXAMPLE 1

### Pyrrolo[3′,4′:5,6]indolizino[8,7-b]indole-1,3(2H,8H)dione

### Step A: 3-[2-(1H-Pyrrol-2-yl)-1H-indol-3-yl]-2,5-pyrrolidinedione

A mixture of the compound of Preparation A (0.274 mmol), of maleimide (0.548 mmol) and a catalytic amount of SnCl₂ in 15 ml of anhydrous toluene is heated under reflux for 24 hours. After evaporation of the toluene, the resulting residue is purified by chromatography on silica gel (ethyl acetate/cyclohexane:3/7) to yield the expected product.

Melting point: 67-69° C.

IR (KBr): ν_{C═O}=1700, 1780 cm⁻¹; ν_{NH}=3100, 3500 cm⁻¹

Mass spectrum (FAB): 279.10 [M⁺]

### Step B: Pyrrolo[3,4′:5,6]indolizino[8,7-b]indole-1,3(2H,8H)dione

A suspension of the compound of the preceding Step (0.358 mmol) and of palladium black (0.358 mmol) in 5 ml of nitrobenzene is heated under reflux for 8 hours. The crude reaction mixture is cooled to ambient temperature, diluted with cyclohexane (5 ml) and placed on a frit containing a plug (5 to 6 cm) of silica gel. The nitrobenzene is eluted using cyclohexane then a cyclohexane/dichloromethane mixture (95/5). The product of the reaction is eluted using a dichloromethane/methanol/trifluoroacetic acid mixture (10/1/0.05). The resulting solution is concentrated and the residue is dissolved in ethyl acetate. That new solution is washed with a saturated sodium hydrogen carbonate solution, with water and then with a saturated sodium chloride solution, dried over magnesium sulphate, filtered and concentrated to yield the expected product.

Melting point: 218-220° C.

IR (KBr): ν_{C═O}=1710, 1750 cm⁻¹; ν_{NH}=2900-3300 cm⁻¹

Mass spectrum (FAB): 275.07 [M⁺]

### EXAMPLE 2

### 2-Methylpyrrolo[3′,4′:5,6]indolizino[8,7-b]indole-1,3(2H,8H)dione

### Step A: 1-Methyl-3-[2-(1H-pyrrol-2-yl)-1H-indol-3-yl]-2,5-pyrrolidinedione

The expected product is obtained according to the process described in Step A of Example 1, using N-methylmaleimide.

Melting point: 142° C.

IR (KBr): ν_{C═O}=1740, 1770 cm¹; ν_{NH}=3200-3400 cm⁻¹

Mass spectrum (FAB): 294.12 [M+H⁺]

### Step B: 2-Methylpyrrolo[3′ 4′:5,6]indolizino[8,7-b]indole-1,3(2H,8H)dione

The expected product is obtained according to the process described in Step B of Example 1, starting from the compound described in the preceding Step.

Melting point: 226-228° C.

IR (KBr): ν_{C═O}=1700-1750 cm⁻¹; ν_{NH}=3400 cm⁻¹

Mass spectrum (FAB): 290.09 [M+H⁺]

### EXAMPLE 3

### 11-(Benzyloxy)pyrrolo[3′,4′:5,6]indolizino[8,7-b]indole-1,3(2H,8H)-dione

### Step A: 3-[5-(Benzyloxy)-2-(1H-pyrrol-2-yl)-1H-indol-3-yl]-2,5-pyrrolidinedione

The expected product is obtained according to the process described in Step A of Example 1, starting from the compound described in Preparation B.

Melting point: 103-107° C.

IR (KBr): ν_{C═O}=1690, 1740 cm⁻¹; ν_{NH}=3250-3440 cm⁻¹

Mass spectrum (FAB): 386.15 [M+H⁺]

### Step B: 11-(Benzyloxy)pyrrolo[3′,4′:5,6]indolizino[8,7-b]indole-1,3(2H,8H)dione

The expected product is obtained according to the process described in Step B of Example 1, starting from the compound described in the preceding Step.

Melting point: 275° C.

IR (KBr): ν_{C═O}=1710, 1720 cm⁻¹; ν_{NH}=3100-3500 cm⁻¹

Mass spectrum (FAB); 382.12 [M+H⁺]

### EXAMPLE 4

### 11-Hydroxypyrrolo[3′,4′:5,6]indolizino[8,7-b]indole-1,3(2H,8H)-dione

### Step A: 3-[5-Hydroxy-2-(1H-pyrrol-2-yl)-1H-indol-3-yl]-2,5-pyrrolidinedione

A suspension of the compound of Step A of Example 3 (0.259 mmol) and 10% palladium-on-carbon (25 mg) in a mixture of ethyl acetate (5 ml) and methanol (10 ml) is hydrogenated at 1 atmosphere for 24 hours. After filtration of the mixture over Celite, the solid is washed with ethyl acetate and methanol. The filtrate is concentrated, allowing the expected product to be obtained.

Melting point: 178-180° C.

IR (KBr): ν_{C═O}=1700, 1720 cm⁻¹; ν_{NH}=3000-3500 cm⁻¹

Mass spectrum (FAB): 295.09 [M+H⁺]

### Step B: 11-Hydroxypyrrolo[3′,4′:5,6]indolizino[8,7-b]indole-1,3(2H,8H)-dione

The expected product is obtained according to the process described in Step B of Example 1, starting from the compound described in the preceding Step.

Melting point: >275° C.

IR (KBr): ν_{C═O}=1710, 1740 cm⁻¹; ν_{NH, OH}=3000-3300 cm⁻¹

### EXAMPLE 5

### 11-(Benzyloxy)-2-methylpyrrolo[3′,4′:5,6]indolizino[8,7-b]indole-1,3-(2H,8H)-dione

### Step A: 3-[5-(Benzyloxy)-2-(1H-pyrrol-2-yl)-1H-indol-3-yl]-]-methyl-2,5-pyrrolidine-dione

The expected product is obtained according to the process described in Step A of Example 1, starting from the compound described in Preparation B and N-methylmaleimide.

Melting point: 89-94° C.

IR (KBr): ν_{C═O}=1680-1700 cm⁻¹; ν_{NH}=3300-3420 cm⁻¹

Mass spectrum (FAB): 400.17 [M+H⁺]

### Step B: 11-(Benzyloxy)-2-methylpyrrolo[3,4′:5,6]indolizino[8,7-b]indole-1,3(2H,8H)-dione

The expected product is obtained according to the process described in Step B of Example 1, starting from the compound described in the preceding Step.

Melting point: 120° C.

IR (KBr): ν_{C═O}=1680-1700 cm⁻¹; ν_{NH}=3200-3600 cm⁻¹

Mass spectrum (FAB): 396.13 [M+H⁺]

### EXAMPLE 6

### 11-Hydroxy-2-methylpyrolo[3′,4′:5,6]indolizino[8,7-b]indole-1,3-(2H,8H-dione)

### Step A: 3-[5-Hydroxy-2-(1H-pyrrol-2-yl)-1H-indol-3-yl]-1-methyl-2,5-pyrrolidinedione

The expected product is obtained according to the process described in Step A of Example 4, starting from the compound described in Step A of Example 5.

Melting point: 148-154° C.

IR (KBr): ν_{C═O}=1680, 1720 cm⁻¹; ν_{NH, OH}=3300-3400 cm⁻¹

Mass spectrum (FAB): 310.12 [M+H⁺]

### Step B: 1-Hydroxy-2-methylpyrrolo[3,4′:5,6]indolizino[8,7-b]indole-1,3(2H,8H-dione)

The expected product is obtained according to the process described in Step B of Example 1, starting from the compound described in the preceding Step.

Melting point: 192° C.

IR (KBr): ν_{C═O}=1700, 1750 cm⁻¹; ν_{NH, OH}=3350-3420 cm⁻¹

Mass spectrum (FAB): 306.09 [M+H⁺]

### EXAMPLE 7

### 11-Bromopyrrolo[3′,4′:5,6]indolizino[8,7-b]indole-1,3(2H,8H)-dione

### Step A: 3-[5-Bromo-2-(1H-pyrrol-2-yl)-1H-indol-3-yl]-2,5-pyrrolidinedione

The expected product is obtained according to the process described in Step A of Example 1, starting from the compound described in Preparation C.

Melting point: 163° C.

IR (KBr): ν_{C═O}=1720, 1780 cm⁻¹; ν_{NH}=3260-3420 cm⁻¹

Mass spectrum (FAB): 357.01 [M⁺]

### Step B: 11-Bromopyrrolo[3,4′:5,6]indolizino[8,7-b]indole-1,3(2H,8H)-dione

The expected product is obtained according to the process described in Step B of Example 1, starting from the compound described in the preceding Step.

Melting point: >300° C.

IR (KBr): ν_{C═O}=1720 cm⁻¹; ν_{NH}=3200-3440 cm⁻¹

Mass spectrum (FAB): 352.98 [M⁺]

### EXAMPLE 8

### 11-Bromo-2-methylpyrrolo[3′,4′:5,6]indolizino[8,7-b]indole-1,3(2H,8H)-dione

### Step A: 3-[5-Bromo-2-(1H-pyrrol-2-yl)-1H-indol-3-yl]-1-methyl-2,5-pyrrolidinedione

The expected product is obtained according to the process described in Step A of Example 1, starting from the compound described in Preparation C and N-methyl-maleimide.

Melting point: 81° C.

IR (KBr): ν_{C═O}=1750-1790 cm⁻¹; ν_{NH}=3340-3400 cm⁻¹

Mass spectrum (FAB): 371.03 [M⁺]

### Step B: 11-Bromo-2-methylpyrrolo[3,4′:5,6]indolizino[8,7-b]indole-1,3(2H,8H)-dione

The expected product is obtained according to the process described in Step B of Example 1, starting from the compound described in the preceding Step.

Melting point: >300° C.

IR (KBr): ν_{C═O}=1650-1690 cm⁻¹; ν_{NH}=3300-3500 cm⁻¹

Mass spectrum (FAB): 366.99 [M⁺]

### EXAMPLE 9

### 11-Chloropyrrolo[3′,4′:5,6]indolizino[8,7-b]indole-1,3(2H,8H)-dione

### Step A: 3-[5-Chloro-2-(1H-pyrrol-2-yl)-1H-indol-3-yl]-2,5-pyrrolidinedione

The expected product is obtained according to the process described in Step A of Example 1, starting from the compound described in Preparation D.

Melting point: 138-144° C.

IR (KBr): ν_{C═O}=1700, 1780 cm⁻¹; ν_{NH}=3100-3500 cm⁻¹

Mass spectrum (FAB): 316.06 [M+H⁺]

### Step B: 11-Chloropyrrolo[3,4′:5,6]indolizino[8,7-b]indole-1,3(2H,8H)-dione

The expected product is obtained according to the process described in Step B of Example 1, starting from the compound described in the preceding Step.

Melting point: 298-304° C.

IR (KBr) ν_{C═O}=1700, 1710 cm⁻¹; ν_{NH}=3100-3400 cm¹

Mass spectrum (FAB): 310.04 [M+H⁺]

### EXAMPLE 10

### 11-Chloro-2-methylpyrrolo[3′,4′:5,6]indolizino[8,7-b]indole-1,3-(2H,8H)-dione

### Step A: 3-[5-Chloro-2-(1H-pyrrol-2-yl)-1H-indol-3-yl]-1-methyl-2,5-pyrrolidinedione

The expected product is obtained according to the process described in Step A of Example 1, starting from the compound described in Preparation D and N-methyl-maleimide.

Melting point: 92-102° C.

IR (KBr): ν_{C═O}=1690, 1770 cm⁻¹; ν_{NH}=3200-3500 cm⁻¹

Mass spectrum (FAB): 327.08 [M⁺]

### Step B: 1-Chloro-2-methylpyrrolo[3′,4′:5,6]indolizino[8,7-b]indole-1,3(2H,8H)-dione

The expected product is obtained according to the process described in Step B of Example 1, starting from the compound described in the preceding Step.

Melting point: 249° C.

IR (KBr): ν_{C═O}=1690, 1710 cm⁻¹; ν_{NH}=3200-3600 cm⁻¹

Mass spectrum (FAB): 324.05 [M+H⁺]

### EXAMPLE 11

### Tert-butyl 2-methyl-1,3-dioxo-1,2,3,4-tetrahydro-7H-dipyrrolo[3,2-a:3,4-c]carbazole-7-carboxylate

### Step A: Tert-butyl 3-[1-methyl-2,5-dioxo-4-(2-pyrrolyl)-2,5-dihydro-1H-pyrrol-3-yl]-1H-indole-1-carboxylate

2M ethylmagnesium bromide in tetrahydrofuran (1.493 mmol) is added dropwise to a solution, maintained at 0° C., of pyrrole (1.493 mmol) in 3 ml of anhydrous tetrahydrofuran. After the mixture has returned to ambient temperature, a solution of the compound described in Preparation E (0.553 mmol) in 6 ml of anhydrous tetrahydrofuran is added dropwise. After 24 hours' stirring at ambient temperature, the reaction mixture is hydrolysed with an aqueous ammonium chloride solution and then extracted with ethyl acetate. The organic phases are combined, dried over magnesium sulphate, filtered and then concentrated. After purification by means of column chromatography on silica gel (ethyl acetate/cyclohexane/triethylamine: 1/4/1%), the expected product is isolated.

Melting point: 82-83° C.

IR (KBr): ν_{C═O}=1700-1740 cm⁻¹; ν_{NH}=3400 cm⁻¹

### Step B: Tert-butyl 2-methyl-1,3-dioxo-1,2,3,4-tetrahydro-7H-dipyrrolo[3,2-a:3,4-c]carbazole-7-carboxylate

A solution of the compound described in the preceding Step (0.204 mmol) in 10 ml of acetonitrile, maintained in a water bath, is irradiated with a halogen lamp (500 W) for 31 hours. After evaporation of the solvent and purification by means of column chromatography on silica gel neutralised with triethylamine (ethyl acetate/cyclohexane/triethylamine:3/7/1%), the expected product is isolated.

Melting point: 172° C. (decomposition)

IR (KBr): ν_{C═O}=1690, 1740, 1760 cm⁻¹; ν_{NH}=3300 cm⁻¹

Mass spectrum (FAB): 390.14 [M+H⁺]

### EXAMPLE 12

### 2-Methyl-4,7-dihydro-1H-dipyrrolo[3,2-a:3,4-c]carbazole-1,3(2H)-dione

The compound described in Example 11 (0.164 mmol) is dissolved in 40 ml of formic acid. After 16 hours' stirring at ambient temperature, the solution is neutralised by the dropwise addition of triethylamine and then an aqueous sodium hydrogen carbonate solution. The mixture is extracted several times with ethyl acetate. The organic phases are combined and then washed with a saturated aqueous sodium chloride solution, dried over magnesium sulphate, filtered and then concentrated. After purification by column chromatography on silica gel (ethyl acetate/cyclohexane:3/7), the expected product is isolated.

Melting point: 292° C.

IR (KBr): ν_{C═O}=1660, 1740 cm⁻¹; ν_{NH}=3320, 3380 cm⁻¹

Mass spectrum (FAB): 290.09 [M+H⁺]

### EXAMPLE 13

### Tert-butyl 6-methyl-5,7-dioxo-5,6,7,7a-tetrahydroimidazo[1,2-a]-pyrido[3′,2′:4,5]pyrrolo[2,3-c]pyrrolo[3,4-e]pyridine-12(4aH)-carboxylate

### Step A: 3-(1H-Imidazol-1-yl)-1-methyl-4-(1H-pyrrolo[2,3-b]pyridin-3-yl-1H-pyrrole-2,5-dione

The expected product is obtained according to the process described in Step A of Example 11, starting from the compound described in Preparation F and imidazole.

Melting point: 246-248° C.

IR (KBr): ν_{C═O}=1710 cm¹; ν_{NH}=3320-3500 cm⁻¹

Mass spectrum (FAB): 296.11 [M+2H⁺]

### Step B: Tert-butyl 3-[4-(1H-imidazol-1-yl)-1-methyl-2,5-dioxo-2,5-dihydro-1H-pyrrol-3-yl]-1H-pyrrolo[2,3-b]pyridine-1-carboxylate

The expected product is obtained according to the process described in Step B of Preparation E, starting from the compound described in the preceding Step.

Melting point: 144-145° C.

IR (KBr): ν_{C═O}=1720, 1740, 1780 cm⁻¹

Mass spectrum (FAB): 394.15 [M+H⁺]

### Step C: Tert-butyl 6-methyl-5,7-dioxo-5,6,7,7a-tetrahydroimidazo[1,2-a]pyrido-[3′,2′,4,5]pyrrolo[2,3-c]pyrrolo[3,4-e]pyridine-12(4aH)-carboxylate

The expected product is obtained according to the process described in Step B of Example 11, starting from the compound described in the preceding Step.

Melting point: 270° C.

IR (KBr): ν_{C═O}=1720, 1750 cm⁻¹

### EXAMPLE 14

### Tert-butyl 6-methyl-5,7-dioxo-6,7-dihydroimidazo[1,2-a]pyrido[3′,2′:4,5]pyrrolo[2,3-c]pyrrolo[3,4-e]pyridine-12(5H)-carboxylate

Manganese dioxide (0.478 mmol) is added to a solution of the compound of Example 13 (0.081 mmol) in 5 ml of anhydrous dichloromethane. The mixture is stirred at ambient temperature for 12 hours and then filtered over Celite® with dichloromethane and methanol. The expected product is obtained after evaporation of the solvents to dryness.

### EXAMPLE 15

### 6-Methylimidazo[1,2-a]pyrido[3′,2′:4,5]pyrrolo[2,3-c]pyrrolo[3,4-e]-pyridine-5,7(6H,12H)-dione

The expected product is obtained according to the process described in Example 12, starting from the compound described in Example 14.

Melting point: 258° C. (decomposition)

IR (KBr): ν_{C═O}=1710, 1760 cm⁻¹; ν_{NH}=3400-3450 cm⁻¹

Mass spectrum (FAB): 394.15 [M+H⁺]

### EXAMPLE 16

### Tert-butyl 2-methyl-1,3-dioxo-2,3,3a,12c-tetrahydroimidazo[1,5-a]-pyrido[3′,2′:4,5]pyrrolo[2,3-c]pyrrolo[3,4-e]pyridine-8(1H)-carboxylate

The expected product is obtained according to the process described in Step B of Example 11, starting from the compound of Preparation F.

Melting point, 152° C.

IR (KBr): ν_{C═O}=1720, 1750 cm⁻¹

### EXAMPLE 17

### Tert-butyl 2-methyl-1,3-dioxo-2,3-dihydroimidazo[1,5-a]pyrido[3′,2′:4,5]pyrrolo[2,3-c]pyrrolo[3,4-e]pyridine-8(1H)-carboxylate

The expected product is obtained according to the process described in Example 14, starting from the compound described in Example 16.

### EXAMPLE 18

### 2-Methylimidazo[1,5-a]pyrido[3′,2′:4,5]pyrrolo[2,3-c]pyrrolo[3,4-e]-Pyridine-1,3(2H,8H)-dione

The expected product is obtained according to the process described in Example 12, starting from the compound described in Example 17.

Melting point: 304-307° C.

IR (KBr): ν_{C═O}=1710, 1760 cm⁻¹ ν_{NH}=3450 cm⁻¹

Mass spectrum (FAB): 292.08 [M^{+H}⁺]

### EXAMPLE 19

### 6-Methyl-7a,12-dihydroimidazo[1,2-a]pyrido[3′,2′:4,5]pyrrolo[2,3-c]-pyrrolo[3,4-e]pyridine-5,7(4aH,6H)-dione

A solution of the compound described in Step B of Example 13 (0.254 mmol) in 6 ml of acetonitrile is irradiated with a halogen lamp (500 W) for 6.5 hours. After evaporation of the solvent and purification by column chromatography on silica gel neutralised with triethylamine (tetrahydrofuran/toluene/triethylamine:3/7/1% to tetrahydrofuran), the expected product is isolated.

Melting point: 222-224° C.

IR (KBr): ν_{C═O}=1710, 1790 cm⁻¹; ν_{NH}=3480 cm⁻¹

Mass spectrum (FAB): 294.10 [M+H⁺]

### EXAMPLE 20

### 2-Methyl-8-(2,3,4,6-tetra-O-acetyl-β-D-glucopyranosyl)-8,12c-dihydroimidazo[1,5-a]pyrido[3′,2′:4,5]pyrrolo[2,3-c]pyrrolo[3,4-e]pyridine-1,3(2H,3aH)-dione

### Step A: 3-(1H-Imidazol-1-yl)-1-methyl-4-[1-(2,3,4,6-tetra-O-acetyl-β-D-glucopyranosyl)-1H-pyrrolo[2,3-b]pyridin-3-yl]-1H-pyrrole-2,5-dione

2,3,4,6-Tetra-O-acetylglucopyranose (0.756 mmol) and triphenylphosphine (0.756 mmol) are added to a solution of the compound described in Step A of Example 13 (0.341 mmol) dissolved in 11 ml of dry tetrahydrofuran. The reaction mixture is cooled to −78° C., and then DEAD (0.756 mmol) is added dropwise. The temperature is slowly raised to ambient temperature, and the reaction mixture is then stirred for a further 15 hours. After hydrolysis, the organic product is extracted with ethyl acetate. The organic phases are combined, dried over magnesium sulphate and filtered, and the solvent is evaporated off. After purification by chromatography on silica gel (cyclohexane/ethyl acetate:7/3 to ethyl acetate), the expected product is isolated.

Melting point: 88-90° C.

IR (KBr): ν_{C═O}=1710, 1750 cm⁻¹

### Step B: 2-Methyl-8-(2,3,4,6-tetra-O-acetyl-β-D-glucopyranosyl)-8,12c-dihydroimidazo-[1,5-a]pyrido[3′,2′:4,5]pyrrolo[2,3-c]pyrrolo[3,4-e]pyridine-1,3(2H,3aH)dione

A solution of the compound obtained in the preceding Step (0.208 mmol) in 10 ml of acetonitrile, maintained in a water bath, is irradiated with a halogen lamp (500 W) for 6 hours. After evaporation of the solvent and purification by column chromatography on silica gel (ethyl acetate/cyclohexane:3/7 to ethyl acetate), the expected product is isolated.

### EXAMPLE 21

### 2-Methyl-8-(2,3,4,6-tetra-O-acetyl-β-D-glucopyranosyl)-imidazo-[1,5-a]pyrido[3′,2′:4,5]pyrrolo[2,3-c]pyrrolo[3,4-e]pyridine-1,3(2H,8H)-dione

The expected product is obtained according to the process described in Example 14, starting from the compound described in Example 20.

Melting point: 204° C.

IR (KBr): ν_{C═O}=1710, 1720, 1750, 1760 cm⁻¹

Mass spectrum (FAB): 622.18 [M+H⁺]

### EXAMPLE 22

### 2-Methyl-8-(β-D-glucopyranosyl)-imidazo[15-a]pyrido[3′,2′:4,5]pyrrolo[2,3-c]pyrrolo[3,4-e]pyridine-1,3(2H,8H)-dione

A 1N solution of sodium methoxide (20 μl) is added dropwise to a solution of the compound described in Example 21 (0.032 mmol) in 6 ml of anhydrous methanol. The mixture is stirrred at ambient temperature for 12 hours. The solvent is evaporated to dryness and the solid is washed on a frit with methanol, allowing the expected product to be isolated.

Melting point: >300° C.

IR (KBr): ν_{C═O}=1710, 1720 cm⁻¹; V_{NH, OH}=3240-3600 cm⁻¹

Mass spectrum (FAB): 454.14 [M+H⁺]

### EXAMPLE 23

### 6-Methyl-12-(2,3,4,6-tetra-O-acetyl-β-D-glucopyranosyl)-7a,12-dihydroimidazo[1,2-a]pyrido[3′,2′:4,5]pyrrolo[2,3-c]pyrrolo[3,4-e]pyridine-5,7(4aH,6H)-dione

The expected product is obtained according to the process described in Step B of Example 20.

### EXAMPLE 24

### 6-Methyl-12-(2,3,4,6-tetra-O-acetyl-β-D-glucopyranosyl)-imidazo-[1,2-a]pyrido[3′,2′:4,5]pyrrolo[2,3-c]pyrrolo[3,4-e]pyridine-5,7(6H,12H)-dione

The expected product is obtained according to the process described in Example 14, starting from the compound described in Example 23.

Mass spectrum (FAB): 622.18 [M+H^{+]}

### EXAMPLE 25

### 6-Methyl-12-(β-D-glucopyranosyl)-imidazo[1,2-a]pyrido[3′,2′:4,5]pyrrolo[2,3-c]pyrrolo[3,4-e]pyridine-5,7(6H,12H)-dione

The expected product is obtained according to the process described in Example 22, starting from the compound described in Example 24.

Melting point: 298° C.

IR (KBr): ν_{C═O}=1710, 1720 cm⁻¹; V_{NH, OH}=3240-3600 cm⁻¹

### EXAMPLE 26

### Pyrido[3′,2′:4,5]pyrrolo[3,2-g]pyrrolo[3,4-e]indolizine-1,3(2H,8H)-dione

### Step A: 3-[2-(1H-Pyrrol-2-yl)-1H-pyrrolo[2,3-b]pyridin-3-yl]-2,5-pyrrolidinedione

A mixture, placed under argon, of the compound of Preparation G (0.546 mmol) and maleimide (5.46 mmol) in a water/methanol solution:2/1 is heated at 50° C. for 48 hours. The methanol is then evaporated off and a saturated aqueous sodium chloride solution is added to the mixture. The reaction mixture is extracted several times with ethyl acetate. The organic phase is dried over magnesium sulphate, filtered and evaporated. Purification by column chromatography on silica gel (ethyl acetate/cyclohexane:1/1 to 1.5/1) yields the expected product.

Melting point: >200° C. (decomposition)

IR (KBr): ν_{C═O}=1700, 1770 cm⁻¹; ν_{NH}=3300-3600 cm⁻¹

### Step B: Pyrido[3′,2′:4,5]pyrrolo[3,2-g]pyrrolo[3,4-e]indolizine-1,3(2H,8H)dione

A suspension of the compound of the preceding Step (0.295 mmol) and of palladium black (0.295 mmol) in 5 ml of nitrobenzene is heated under reflux for 7 hours. The reaction mixture is filtered over silica gel, eluted with dichloromethane and then with tetrahydrofuran. Purification by column chromatography on silica gel (tetrahydrofuran/dichloromethane:1/9 then 2/8) yields the expected product.

Melting point: >300° C. (decomposition)

IR (KBr): ν_{C═O}=1720, 1760 cm⁻¹; ν_{NH}=3150-3300 cm⁻¹

### Pharmacological Study of Compounds of the Invention

### EXAMPLE 27

### In Vitro Activity

Four cell lines were used:
L1210 murine leukaemia
A549 human non-small-cell lung carcinoma
HT29 human colon carcinoma
DU145 prostate carcinoma

L1210 murine leukaemia was used in vitro. The cells are cultured in RPMI 1640 complete culture medium containing 10% foetal calf serum, 2 mM glutamine, 50 units/ml of penicillin, 50 μg/ml of streptomycin and 10 mM Hepes, pH: 7.4. The cells are distributed on microplates and are exposed to the cytotoxic compounds for 4 doubling periods, or 48 hours. The number of viable cells is then quantified by a calorimetric assay, the Microculture Tetrazolium Assay (J. Carmichael et al., Cancer Res.; 47, 936-942 (1987)). The results are expressed as the IC₅₀, the concentration of cytotoxic agent which inhibits the proliferation of the treated cells by 50%. By way of example, the compound of Example 1 exhibits IC₅₀ values of 3.1 μM on L1210, 1.99 μM on A549, 3.3 μM on HT29 and 1.4 μM on DU145.

### EXAMPLE 28

### Pharmaceutical Composition: Injectable Solution

| | | |
|---|---|---|
| Compound of Example 9 | 10 | mg |
| Distilled water for injectable preparations | 25 | ml |

1-21. (canceled)

22. A compound selected from those of formula (I): 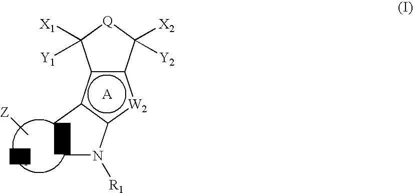
wherein:
A represents a saturated or partially or fully unsaturated 6 membered ring, wherein the unsaturation optionally confers aromaticity on the ring,
Z represents one or more identical or different groups of formula U-V wherein:
U represents a single bond, linear or branched (C₁-C₆)alkylene optionally substituted by one or more identical or different groups selected from halogen and hydroxy and/or optionally containing one or more unsaturated bonds,
V represents a group selected from hydrogen, halogen, cyano, nitro, azido, linear or branched (C₁-C₆)alkyl, aryl, aryl(C₁-C₆)alkyl in which the alkyl moiety may be linear or branched, hydroxy, linear or branched (C₁-C₆)alkoxy, aryloxy, aryl(C₁-C₆)alkoxy in which the alkoxy radical may be linear or branched, formyl, carboxy, aminocarbonyl, NR₃R₄, —C(O)-T₁, —C(O)—NR₃-T₁, —NR₃—C(O)-T₁, —O—C(O)-T₁, —C(O)—O-T₁, —O-T₂-NR₃R₄, —O-T₂-OR₃, —O-T₂-CO₂R₃, —NR₃-T₂-NR₃R₄, —NR₃-T₂-OR₃, —NR₃-T₂-CO₂R₃, and —S(O)ₜ—R₃, wherein:
R₃ and R₄, which may be identical or different, each represents a group selected from hydrogen, linear or branched (C₁-C₆)alkyl, aryl, and aryl(C₁-C₆)alkyl in which the alkyl moiety may be linear or branched, or
R₃+R₄ together with the nitrogen atom carrying them, form a saturated monocyclic or bicyclic heterocycle that has from 5 to 10 ring atoms, and which optionally contains in the ring system a second hetero atom selected from oxygen and nitrogen, and which is optionally substituted by a group selected from linear or branched (C₁-C₆)alkyl, aryl, aryl(C₁-C₆)alkyl in which the alkyl moiety may be linear or branched, hydroxy, linear or branched (C₁-C₆)alkoxy, amino, linear or branched mono(C₁-C₆)alkylamino, and di(C₁-C₆)alkylamino in which the alkyl moieties may be linear or branched,
T₁ represents a group selected from linear or branched (C₁-C₆)alkyl which may be optionally substituted by a group selected from —OR₃, —NR₃R₄, —CO₂R₃, —C(O)R₃ and —C(O)NR₃R₄ wherein R₃ and R₄ are as defined hereinbefore; aryl, and aryl(C₁-C₆)alkyl in which the alkyl moiety may be linear or branched; or T₁ represents linear or branched (C₂-C₆)alkenyl optionally substituted by a group selected from —OR₃, —NR₃R₄, —CO₂R₃, —C(O)R₃ and —C(O)NR₃R₄ wherein R₃ and R₄ are as defined hereinbefore,
T₂ represents linear or branched (C₁-C₆)alkylene,
t represents integer from 0 to 2 inclusive,
or Z represents methylenedioxy or ethylenedioxy,
W₁, together with the carbon atoms to which it is bonded, represents phenyl or pyridyl,
W₂ represents a group selected from: 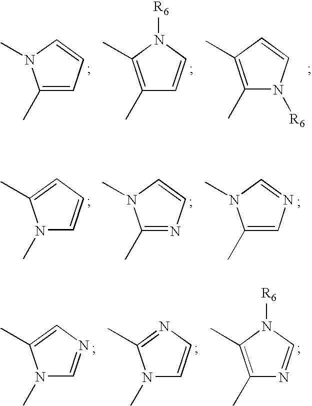
wherein R₆ represents a group selected from hydrogen, linear or branched (C₁-C₆)alkyl, aryl, aryl(C₁-C₆)alkyl in which the alkyl moiety may be linear or branched, cycloalkyl, cycloalkyl(C₁-C₆)alkyl in which the alkyl moiety may be linear or branched, —OR₃, —NR₃R₄, —O-T₂-NR₃R₄, —NR₃-T₂-NR₃R₄, linear or branched (C₁-C₆)hydroxyalkylamino, di((C₁-C₆)hydroxyalkyl)amino in which the alkyl moieties may be linear or branched, —C(O)R₃ and —NH—C(O)R₃; or R₆ represents linear or branched (C₁-C₆)alkylene substituted by one or more identical or different groups selected from halogen, cyano, nitro, —OR₃, —NR₃R₄, —CO₂R₃, —C(O)R₃, linear or branched (C₁-C₆)hydroxyalkylamino, di((C₁-C₆)hydroxyalkyl) amino in which the alkyl moieties may be linear or branched, and —C(O)—NHR₃, R₃, R₄ and T₂ being as defined hereinbefore,
X₁ represents a group selected from hydrogen, hydroxy, linear or branched (C₁-C₆)alkoxy, mercapto and linear or branched (C₁-C₆)alkylthio,
Y₁ represents hydrogen, or
X₁ and Y₁, with the carbon atom carrying them, form carbonyl or thiocarbonyl,
X₂ represents a group selected from hydrogen, hydroxy, linear or branched (C₁-C₆)alkoxy, mercapto and linear or branched (C₁-C₆)alkylthio,
Y₂ represents hydrogen, or
X₂ and Y₂, with the carbon atom carrying them, form carbonyl or thiocarbonyl group,
R₁ represents a group selected from hydrogen, a linear or branched (C₁-C₆)alkyl which may be optionally substituted by one or more groups selected from hydroxy, linear or branched (C₁-C₆)alkoxy, linear or branched (C₁-C₆)hydroxyalkoxy or NR₃R₄, the groups R₃ and R₄ being as defined hereinbefore; or R₁ represents a group of formula C(O)—O-T₃ wherein T₃ represents a group selected from linear or branched (C₁-C₆)alkyl, aryl and aryl(C₁-C₆)alkyl in which the alkyl moiety may be linear or branched; or R₁ represents a group of formula (a): 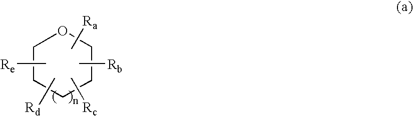
wherein:
Rₐ, R_{b}, R_{c} and R_{d}, which may be identical or different, each represents, independently of the others, a bond or a group selected from hydrogen, halogen, hydroxy, linear or branched (C₁-C₆)alkoxy, aryloxy, aryl(C₁-C₆)alkoxy in which the alkoxy moiety may be linear or branched, linear or branched (C₁-C₆)alkyl, aryl(C₁-C₆)alkyl in which the alkyl moiety may be linear or branched, aryl, —NR₃R₄ wherein R₃ and R₄ are as defined hereinbefore, azido, —N═NR₃ (wherein R₃ is as defined hereinbefore), and —O—C(O)R₅ wherein R₅ represents linear or branched (C₁-C₆)alkyl (optionally substituted by one or more groups selected from halogen, hydroxy, amino, linear or branched (C₁-C₆)alkylamino, and di(C₁-C₆)alkylamino in which the alkyl moieties may be linear or branched); or R₅ represents aryl, aryl(C₁-C₆)alkyl in which the alkyl moiety may be linear or branched, cycloalkyl or heterocycloalkyl,
Rₑ represents methylene (H₂C═) or a group of the formula —U₁—Rₐ wherein U₁ represents single bond, methylene and Rₐ is as defined hereinbefore,
n is 0 or 1,
it being understood that the group of formula (a) is bonded to the nitrogen atom by Rₐ, R_{b}, R_{c}, R_{d} or Rₑ,
Q represents a group selected from oxygen, NR₂ wherein R₂ represents a group selected from hydrogen, linear or branched (C₁-C₆)alkyl, aryl, aryl(C₁-C₆)alkyl in which the alkyl moiety may be linear or branched, cycloalkyl, cycloalkyl(C₁-C₆)alkyl in which the alkyl moiety may be linear or branched, —OR₃, —NR₃, —O-T₂-NR₃R₄, —NR₃-T₂-NR₃R₄, linear or branched (C₁-C₆)hydroxyalkylamino, di((C₁-C₆)hydroxyalkyl)amino in which the alkyl moieties may be linear or branched, —C(O)—R₃ and —NH—C(O)—R₃; or R₂ represents linear or branched (C₁-C₆)alkylene substituted by one or more identical or different groups selected from halogen, cyano, nitro, —OR₃, —NR₃R₄, —CO₂R₃, —C(O)R₃, linear or branched (C₁-C₆)hydroxyalkylamino, di((C₁-C₆)hydroxyalkyl)amino in which the alkyl moieties may be linear or branched, and —C(O)NHR₃, R₃, R₄ and T₂ being as defined hereinbefore,
it being understood that:
when W_{1,} together with the carbon atoms to which it is bonded, represents an unsubstituted phenyl or phenyl substituted by bromine, R₁ represents a group selected from hydrogen, and glucopyranosyl or (2,3,4,6-tetra-O-benzyl-glucopyranosyl) and, R₂ represents hydrogen, then W₂ represents a group selected from: 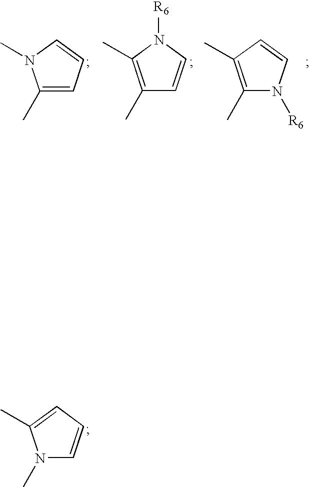
wherein R₆ is as defined hereinbefore,
when W₁, together with the carbon atoms to which it is bonded, represents an unsubstituted phenyl, R₁ represents hydrogen and R₂ represents methyl, then W₂ represents a group selected from: 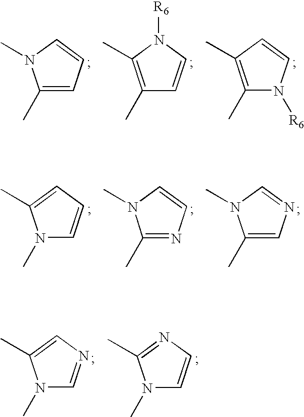
wherein R₆ is as defined hereinbefore,
and aryl may be a phenyl, naphthyl, dihydronaphthyl, tetrahydronaphthyl, indenyl or indanyl group, each of those groups optionally being substituted by one or more identical or different substituents selected from halogen, linear or branched (C₁-C₆)alkyl, linear or branched (C₁-C₆)trihaloalkyl, hydroxy, linear or branched (C₁-C₆)alkoxy, and NR₃R₄, wherein R₃ and R₄ are as defined hereinbefore.

23. A compound of claim 22, wherein X₁ and Y₁, with the carbon atom carrying them, together form carbonyl, and X₂ and Y₂, with the carbon atom carrying them, together form carbonyl.

24. A compound of claim 22, wherein Q represents —NR₂.

25. A compound of claim 22, which is a compound of formula (IA):

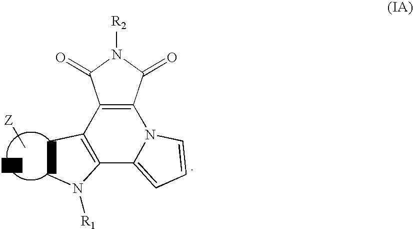

26. A compound of claim 22, which is a compound of formula (IB):

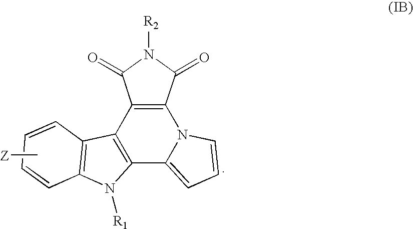

27. A compound of claim 22, which is a compound of formula (IC):

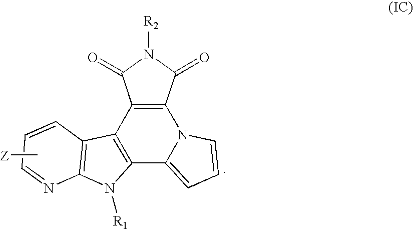

28. A compound of claim 22, which is a compound of formula (ID):

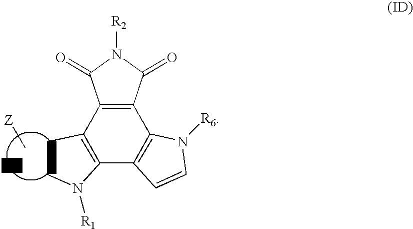

29. A compound of claim 22, which is a compound of formula (IE):

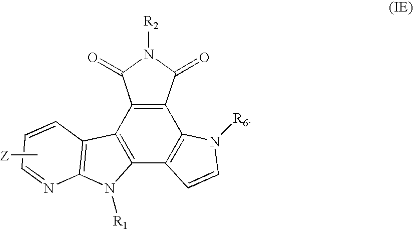

30. A compound of claim 22, which is a compound of formula (IF):

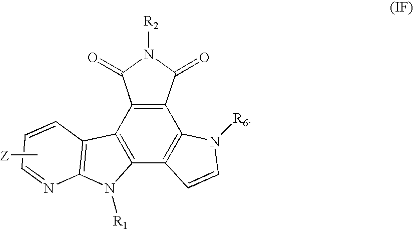

31. A compound of claim 22, which is a compound of formula (IG):

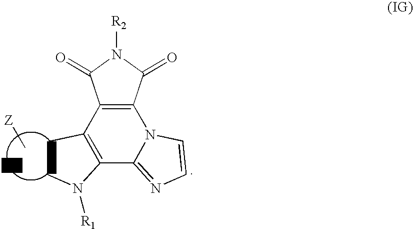

32. A compound of claim 22, which is a compound of formula (IH):

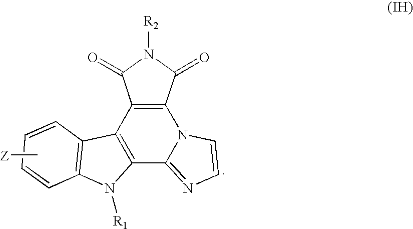

33. A compound of claim 22, which is a compound of formula (II):

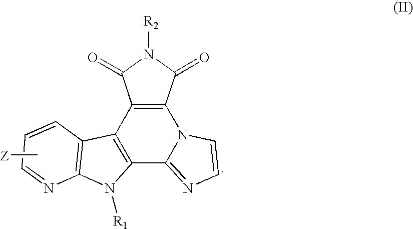

34. A compound of claim 22, which is a compound of formula (IJ):

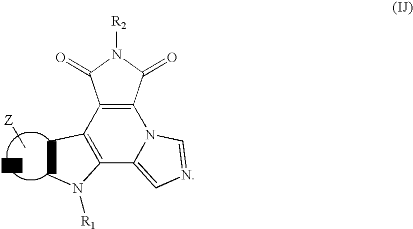

35. A compound of claim 22, which is a compound of formula (IK):

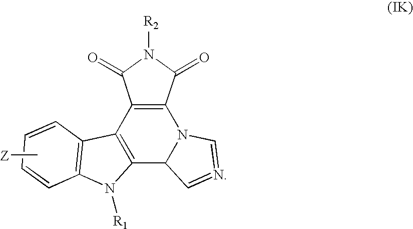

36. A compound of claim 22, which is a compound of formula (IL):

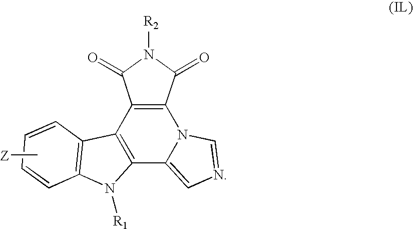

37. A compound of claim 22, wherein R₁ represents hydrogen, C(O)—O-T₃ wherein T₃ represents linear or branched (C₁-C₆)alkyl or a glucopyranosyl group of formula:

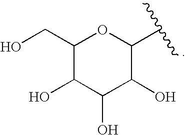

38. A compound of claim 22, wherein R₂ represents hydrogen or linear or branched (C₁-C₆) alkyl.

39. A compound of claim 22, wherein R₆ represents hydrogen.

40. A compound of claim 22 which is selected from:
pyrrolo[3′,4′:5,6]indolizino[8,7-b]indole-1,3[2H,8H]-dione,
11-bromopyrrolo[3′,4′:5,6]indolizino[8,7-b]indole-1,3 [2H,8H]-dione,
11-chloropyrrolo[3′,4′:5,6]indolizino[8,7-b]indole-1,3 [2H,8H]-dione, and
imidazo[2′,1′:6,1]pyrrolo[3′,4′:4,5]pyrido[2,3-b]indole-1,3(2H,8H)-dione.

41. A method for treating a living animal body, including a human, afflicted with cancer comprising the step of administering to the living body, including a human an amount of a compound of claim 22, which is effective for alleviation of cancer

42. A pharmaceutical composition useful in treating cancer comprising as active principle an effective amount of a compound of claim 22, together with one or more pharmaceutically acceptable excipients or vehicles.
